(19) United States
(12) Patent Application Publication
(10) Pub. No.: US 2002/0153126 A1
(43) Pub. Date: Oct. 24, 2002

(54) Multi-functional mobile climate control assembly for personal use

(76) Inventor: Roger Clemente, Kenilworth, NJ (US)

Correspondence Address: CLIFFORD G. FRAYNE, Suite 7A, 136 Drum Point Road., Brick, NJ, 08723, US

(21) Appl. No.: 10/114,180

(22) Filed: Apr. 2, 2002

Related U.S. Application Data
(60) Provisional application No. 60/282,000, filed on Apr. 9, 2001.

(51) Int. Cl. F25B 21/02; F28F 7/00; A61F 7/00; F25D 23/12

(52) U.S. Cl. 165/46; 62/3.5; 62/259.3; 607/104; 607/108

(57) ABSTRACT

An improved, multi-functional, portable, electronic climate control device to maintain personal body temperature at a desired level in relationship to different environments. The portable electronic control device can combine a refrigeration system with a circulation pump in fluid communication with a garment-like vest to provide personal cooling below surrounding ambient temperature, or include an electronic heating element with circulating pump in fluid communication with a garment-like vest to maintain personal body temperature above surrounding environment.

### RELATED APPLICATIONS

Applicant claims the benefit of the filing date of provisional application Serial No. 60/282,000, filed Apr. 9, 2001.

### BACKGROUND OF THE INVENTION

1. Field of the Invention

The present invention relates to a climate control assembly and more particularly to a garment for personal wear which can be cooled or heated by the proximately located assembly.

2. Description of the Prior Art

The environment in which we live, work and play challenges the methods used to control body temperature. Within your home, office or automobile, the heating and air conditioning systems can be manipulated to maintain a cool environment in summer and a warm environment in the winter. However, in open air environments subject to the cold of winter and the heat of summer, there is no effective way for an individual to maintain a comfortable body temperature. This is particularly true in work situations where people must wear protective clothing (military personnel, race car driver, haz-mat worker, unprotected construction machine operators, and the like). Their body temperatures are simply maintained at a comfortable level by adding clothing or discarding clothing, depending on whether the worker is subjected to the cold of winter or the heat of summer.

Attempts have been made to devise apparatus which would provide for individual cooling. Up to now these devices have consisted of jackets, vests or pants having receptacles or pockets in which raw ice is deposited in the heat of summer to cool the body. In some instances, a packet containing a frozen solvent is inserted into these pouches or receptacles. These have met with some degree of success, but are not widely accepted since the ice or the ice packet has to constantly be replenished. This sometimes proves difficult at a work site or remote location. Further, such devices are not appropriate when working in harsh, toxic environments where uninterrupted cooling for extended periods is a requirement for life support. Applicant's invention is light weight and highly effective and lends itself to use by an individual who has to perform tasks within a relatively limited area.

### OBJECTS OF THE INVENTION

An object of the present invention is to provide for a novel climate control apparatus adapted for individual personal use.

A still further object of the present invention is to provide for a novel climate control apparatus which is light weight and portable and can be powered by an inverter, transformer, battery, equivalent photo electric cell, or wind generator, etc.

A still further object of the present invention is to provide for a novel personal climate control apparatus in which the user can adjust the temperature to the desired amount.

A still further object of the present invention is to provide for a novel climate control apparatus which permits the user a range of mobility.

A still further object of the present invention is to provide for a novel climate control apparatus which provides the user with a quick release mechanism so as to disengage the garment from the cooling apparatus.

A still further object of the present invention is to provide for a novel individual climate control apparatus in which a heating element is interposed to accomplish the objects heretofore set forth and to warm the user.

### SUMMARY OF THE INVENTION

A battery operated multi-functional climate control assembly and garment in which the individual user wears the garment on his body, the garment having an inlet port and an outlet port and a plurality of fluid passageways formed in the garment between the inlet port and the outlet port, a temperature control unit comprising a vapor compressor in communication with an evaporator, the evaporator being in communication with the garment by means of an inlet conduit and an outlet conduit, there also being a container for maintaining fluid in the system, and a pump to move the fluid from the evaporator to the garment and return the fluid from the garment to the evaporator, the conduit lines being of sufficient length to allow the operator a degree of mobility. Alternatively if there was a desire for the individual to heat his body, the vapor compressor can be disengaged and water from the evaporator reservoir would pass through an electric heater before being directed through the conduits to the garment.

### BRIEF DESCRIPTION OF THE DRAWINGS

These and other objects of the present invention will become evident, particularly when taken in light of the following illustrations wherein:

FIG. 1 is an exploded view of the overall personal, individual, cooling control assembly.

FIG. 2 is an exploded view of a roll bond evaporator assembly and vapor compressor of the present invention.

FIG. 3 is an exploded view of the personal cooling control assembly utilized as a heating assembly.

FIG. 4 is an open view of a suitable garment to be utilized with the personal cooling control assembly.

### DETAILED DESCRIPTION OF THE DRAWINGS

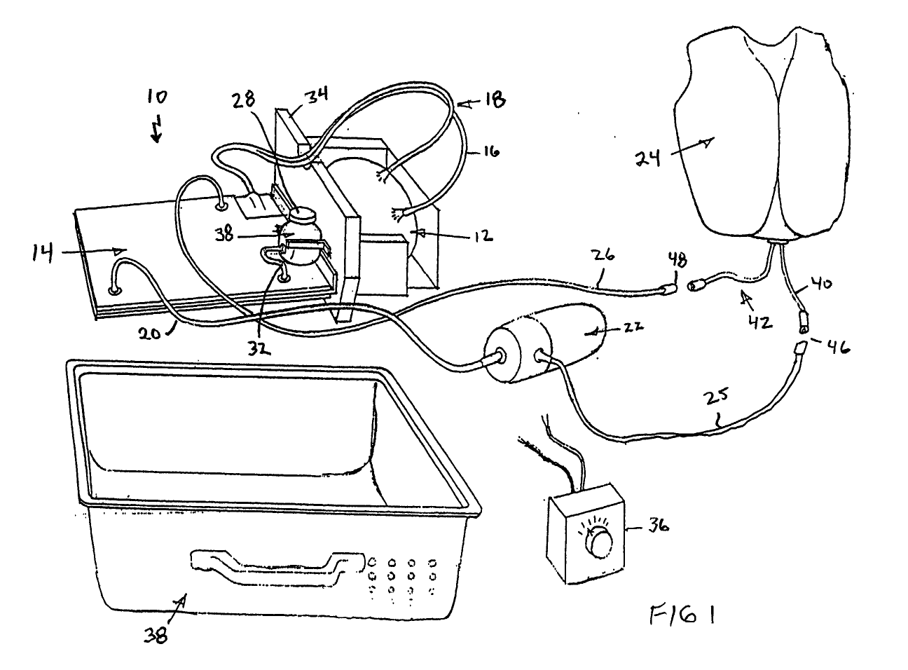

FIG. 1 is an exploded view of the cooling control assembly 10. Cooling control assembly 10 includes a vapor compressor 12 containing a suitable fluid refrigerant. Vapor compressor 12 is in fluid communication with an evaporator 14 by means of refrigerant lines 16 and 18, one line being for communication of fluid refrigerant to evaporator 14 and one line being a return line. Evaporator 14 is in fluid communication via conduit 20 with a pump means 22 which in turn is in fluid communication via conduit 25 with a garment 24 illustrated in FIG. 1 as a vest. A return conduit 26 serves to return the fluid to the evaporator. The evaporator 14 also has associated therewith a fill spout 28 and fluid reservoir 30 in communication with the evaporator 14 by means of conduit 32. Evaporator compartment housing 14A is filled with a suitable fluid such as a mixture of water and alcohol similar to that of the windshield washer fluid utilized in automobiles. The vapor compressor 12 is separated from evaporator 14 by means of an insulation wall 34.

A thermostat 36 may be incorporated in the system to control the temperature of the fluid circulating between the evaporator 14 and the garment 24. A circulating liquid valve (not shown) may also be installed in line 25 or 26 as an alternative means to control temperature by adjusting the cooling liquid flow through the vest garment.

It is intended that the vapor compressor 12, evaporator 14, pump 22, fill spout 30, and reservoir 32, together with the thermostat 36 be housed in a hand held carrying case 38 with the conduit lines 25 and 26 being of sufficient length to allow the user to maneuver at his particular job site by distance from the assembly 10 in carrying case 38.

Further, conduits 25 and 26 are secured to conduits 40 and 42 emanating from the garment 24 by quick release fittings 46 and 48. This allows the user in case of emergency to quickly disconnect the garment from the apparatus should the user have to move rapidly or move a distance beyond the length of conduits 25 and 26. The quick release fittings 46 and 48 automatically seal the ends of the conduits so there is a minimal loss of fluid from the system.

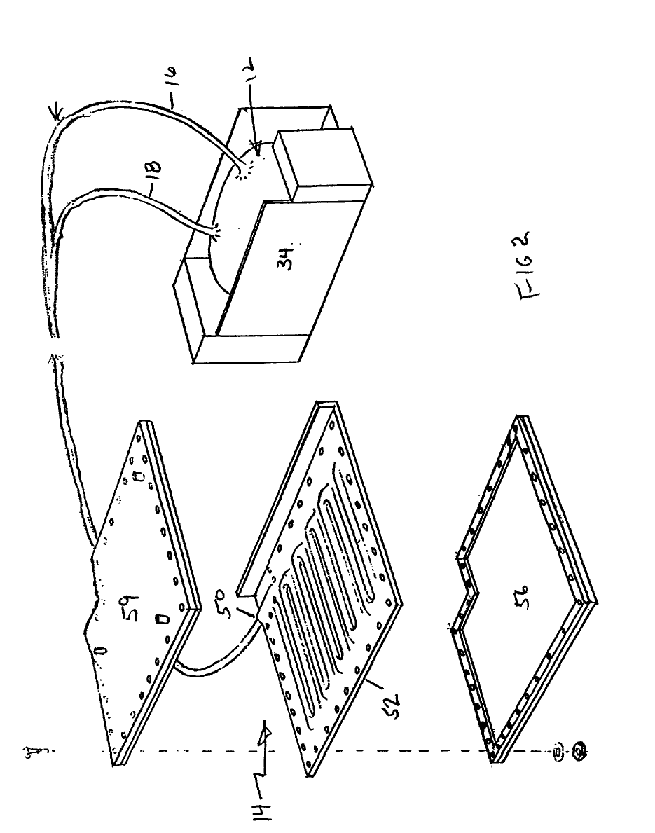

FIG. 2 is an exploded view of evaporator 14 which in the preferable embodiment would be a braze plate or roll bond evaporator in which the fluid refrigerant from the vapor compressor 12 would be introduced into a closed loop circuit 50 formed on a central panel 52 which would be sandwiched between an upper closure member 54 and a lower closure member 56 with the fluid to be cooled circulating above and below the closed loop circuit 50.

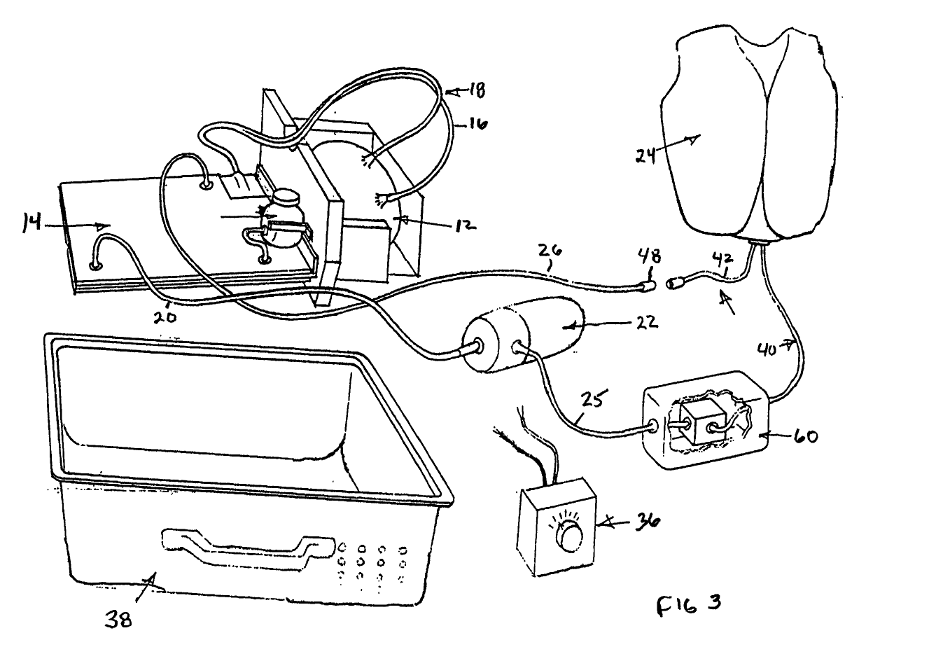

FIG. 3 is another embodiment of assembly 10 as illustrated in FIG. 1 adapted to be utilized to circulate heated fluid through the garment 24 under cold weather conditions. In this instance, the vapor compressor 12 would be disconnected from the evaporator 14. An electrical heating element 60 would be positioned in the conduit line 20 or 25 leading from the evaporator to the garment 24. The electric heating element 60 would again be controlled by a thermostat 36 to adjust the temperature of the fluid circulating between the evaporator and the garment 24.

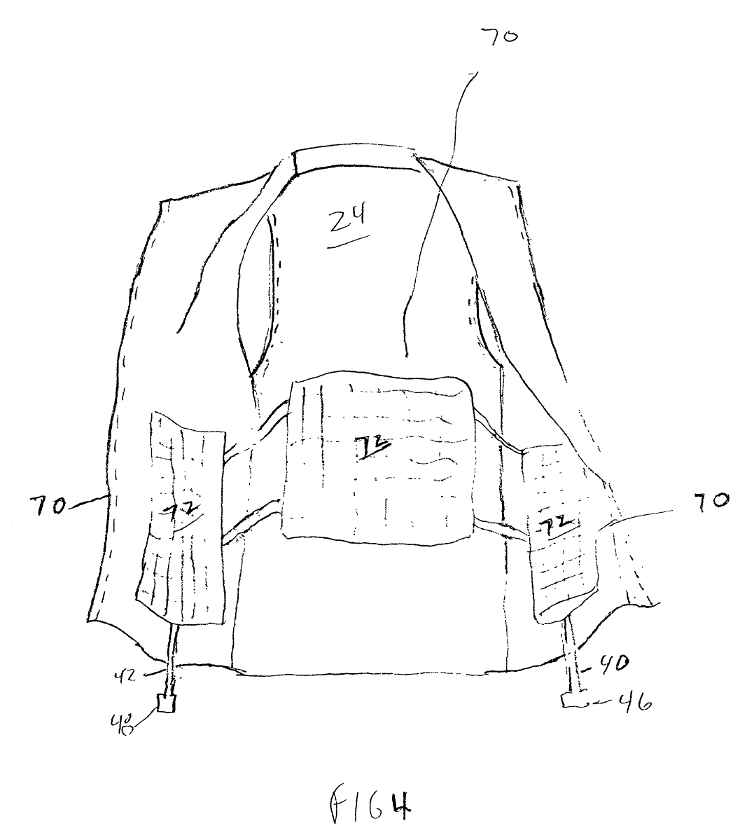

FIG. 4 is an open view of garment 24 illustrating an embodiment of the manner in which panels 70 having honeycombed passageways 72 could be secured within the inner surface of the garment in order to provide either a cooling or a heating affect to the torso of the individual. Fluid would enter via conduit 40 and in steady state operation would flow through the honeycomb passageways 72 of a first panel before flowing to a second panel and subsequently a third panel where the fluid would be returned via conduit 42 to the evaporator. FIG. 4 illustrates a vest with three panels, however, a choice of the number of panels and their positioning would be one of choice and would be dependent on the extreme conditions of the ambient environment.

The power source for the assembly 10 could be an ordinary car battery if the individual utilizing the system were operating a vehicle. The system could be adapted to plug into the cigarette lighter or other suitably designed receptacle. In a non-vehicle situation the assembly 10 could be operated by a 12 volt battery or a photoelectric cell.

While the design is directed to a garment for individual use, it will be recognized that in certain expedient circumstances, the garment itself may be utilized to wrap around an object in order to maintain the object at a desired cooler or hotter temperature than the ambient atmosphere.

What is claimed:

1. A portable, electronic climate control device that provides a personal body temperature environment below the surrounding ambient temperature comprising:
a refrigeration vapor compressor;
an evaporator assembly in communication with said refrigeration vapor compressor;
a pump assembly in communication with said evaporator assembly;
a fluid refrigerant;
a garment worn by a user, said garment in communication with said pump assembly and said evaporator assembly, said garment having formed therein a plurality of matrix conduit passageways in fluid communication with each other for the passage therethrough of said fluid refrigerant from said pump, and the return of said fluid refrigerant to said evaporator assembly;
a power source for powering said portable, electronic climate control device.

2. The portable, electronic climate control device in accordance with claim 1 wherein said power source is an inverter, transformer, battery, solar panel, wind turbine or water turbine.

3. A portable, electronic climate control device in accordance with claim 1 wherein said garment worn by said user is designed to fit an upper torso of a user's body.

4. A portable, electronic climate control device in accordance with claim 1 wherein said matrix conduit passageways are disposed on the interior of said garment.

5. A portable, electronic climate control device that provides a personal body temperature environment above surrounding ambient temperature comprising:
a fluid reservoir;
a pump means in communication with said fluid reservoir;
a heating element in communication with said pump means;
a garment worn by a user, said garment having a plurality of matrix fluid communication lines disposed therein, one of said plurality of matrix fluid communication lines in communication with the said heating element, and a separate one of said plurality of matrix fluid communication lines in communication with said fluid reservoir;
a power source.

6. A portable, electronic climate control device in accordance with claim 5 wherein said power source is an inverter, transformer, battery, solar panel, wind turbine or water turbine.

7. A portable, electronic climate control device in accordance with claim 5 wherein said garment worn by said user is designed to fit an upper torso of a user's body.

8. A portable, electronic climate control device in accordance with claim 5 wherein said matrix conduit passageways are disposed on the interior of said garment.
